(19) United States
(12) Patent Application Publication
(10) Pub. No.: US 2022/0237536 A1
(43) Pub. Date: Jul. 28, 2022

(54) RISK ESTIMATION APPARATUS, RISK ESTIMATION METHOD, COMPUTER PROGRAM AND RECORDING MEDIUM

(71) Applicant: NEC Corporation, Minato-ku, Tokyo (JP)

(72) Inventor: Tasuku Kitade, Tokyo (JP)

(73) Assignee: NEC Corporation, Minato-ku, Tokyo (JP)

(21) Appl. No.: 17/617,651

(22) PCT Filed: Mar. 10, 2020
(86) PCT No.: PCT/JP2020/010240

§ 371 Date: Dec. 9, 2021

(30) Foreign Application Priority Data
Jun. 17, 2019 (JP) 2019-111762

(51) Int. Cl. G06Q 10/06; A61B 5/16

(52) U.S. Cl. CPC G06Q 10/0635; A61B 5/165; G06Q 10/06398

(57) ABSTRACT

A risk estimation apparatus includes: an extraction unit that extracts a factor information, which is a statistic of a physical and mental information about a factor that causes a change in work stress of a subject person, from a physical and mental information including at least one of a physiological information of the subject person and an inner state information indicating an inner state of the subject person estimated on the basis of the physiological information; an arithmetic unit that obtains a feature quantity in a predetermined time unit from the factor information; and an estimation unit that estimates a risk degree related to at least one of leave of absence, job separation or productivity of the subject person, by inputting the feature quantity into a predetermined learning model.

### TECHNICAL FIELD

The present invention relates to a risk estimation apparatus, a risk estimation method, a computer program and a recording medium, and, in particular, to a risk estimation apparatus, a risk estimation method, a computer program and a recording medium that estimate a company risk, such as, for example, leave of absence, job separation, and reduced productivity.

### BACKGROUND ART

For a technique used in this type of apparatus, for example, a technique described in Patent Literature 1 has been proposed. That is, in the technique described in Patent Literature 1, a feature vector is generated from features related to an average number or the like of a work place, a job period, different job types and work hours obtained by performing filtering on time series data based on a personnel information on an employee, such as a personal information, a job history, and a promotion of the employee. Then, the feature vector is inputted into a prediction model that predicts a risk of job separation, thereby to predict the risk of job separation. Other related techniques include Patent Literatures 2 to 5 and Non-Patent Literature 1.

### CITATION LIST

### Patent Literature

Patent Literature 1: JP 6246776B
Patent Literature 2: JP2019-13737A
Patent Literature 3: JP2018-171124A
Patent Literature 4: JP2016-207165A
Patent Literature 5: JPH10-80412A

### Non-Patent Literature

Non-Patent Literature 1: Yoshiki Nakashima, Masanori Tsujigawa, and Yoshifumi Onishi, “Improvement in Chronic Stress Level Recognition by Using Both Full-term and Short-term Measurements of Physiological Features”, The 32nd Annual Conference of the Japanese Society for Artificial Intelligence, 2018.

### SUMMARY OF INVENTION

### Technical Problem

In the technique described in Patent Literature 1, it is hardly possible to estimate the risk due to physical and, or psychological burden that is caused by work (i.e., work stress) and that does not appear in the personnel information, which is technically problematic.

In view of the problems described above, it is therefore an example object of the present invention to provide a risk estimation apparatus, a risk estimating method, a computer program and a recording medium that are configured to estimate a risk related to leave of absence, job separation, productivity or the like due to work stress.

### Solution to Problem

A risk estimation apparatus according to an example aspect of the present invention includes: an extraction unit that extracts a factor information, which is a statistic of a physical and mental information about a factor that causes a change in work stress of a subject person, from the physical and mental information including at least one of a physiological information of the subject person and an inner state information indicating an inner state of the subject person estimated on the basis of the physiological information; an arithmetic unit that obtains a feature quantity in a predetermined time unit from the factor information; and an estimation unit that estimates a risk degree related to at least one of leave of absence, job separation or productivity of the subject person, by inputting the feature quantity into a predetermined learning model.

A risk estimation method according to an example aspect of the present invention extracts a factor information, which is a statistic of a physical and mental information about a factor that causes a change in work stress of a subject person, from the physical and mental information including at least one of a physiological information of the subject person and an inner state information indicating an inner state of the subject person estimated on the basis of the physiological information; obtains a feature quantity in a predetermined time unit from the factor information; and estimates a risk degree related to at least one of leave of absence, job separation or productivity of the subject person, by inputting the feature quantity into a predetermined learning model.

A computer program according to an example aspect of the present invention allows a computer to perform the risk estimation method according to the example aspect described above.

A recording medium according to an example aspect of the present invention is a recording medium on which the computer program according to the example aspect described above is recorded.

### Advantageous Effects of Invention

According to the risk estimation apparatus, risk estimation method, computer program and recording medium in the respective example aspects described above, it is possible to estimate the risk related to leave of absence, job separation, productivity or the like due to work stress.

### BRIEF DESCRIPTION OF DRAWINGS

FIG. 1 is a block diagram illustrating a hardware configuration of a risk estimation apparatus according to a first example embodiment.

FIG. 2 is a block diagram illustrating a functional block implemented in a CPU according to the first example embodiment.

FIG. 3 is a conceptual diagram illustrating a specific example of a risk degree estimation unit according to the first example embodiment.

FIG. 4 is a flowchart illustrating the operation of the risk estimation apparatus according to the first example embodiment.

FIG. 5 is a block diagram illustrating a functional block implemented in a CPU according to a second example embodiment.

FIG. 6 is a diagram illustrating an example of a causal loop diagram.

FIG. 7 is a flowchart illustrating the operation of a risk estimation apparatus according to the second example embodiment.

FIG. 8 is a block diagram illustrating a functional block implemented in a CPU according to a modified example.

### DESCRIPTION OF EXAMPLE EMBODIMENTS

A risk estimation apparatus, a risk estimation method, a computer program, and a recording medium according to example embodiments will be described with reference to the drawings. The following describes the risk estimation apparatus, the risk estimation method, the computer program, and the recording medium according to the example embodiment, by using a risk estimation apparatus 1.

### First Example Embodiment

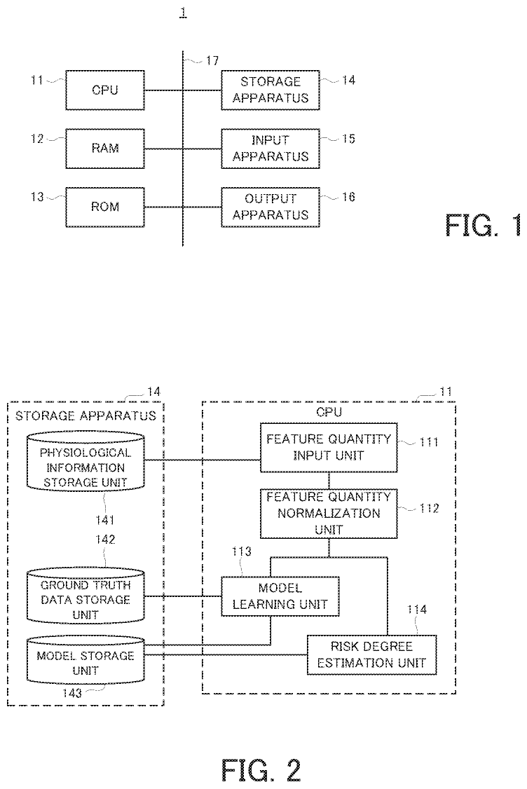

The risk estimation apparatus 1 according to a first example embodiment will be described with reference to FIG. 1 to FIG. 4.

(Configuration)

Firstly, a hardware configuration of the risk estimation apparatus 1 according to the first example embodiment will be described with reference to FIG. 1. FIG. 1 is a block diagram illustrating the hardware configuration of the risk estimation apparatus 1 according to the first example embodiment.

In FIG. 1, the risk estimation apparatus 1 includes a CPU (Central Processing Unit) 11, a RAM (Random Access Memory) 12, a ROM (Read Only Memory) 13, a storage apparatus 14, an input apparatus 15, and an output apparatus 16. The CPU 11, the RAM 12, the ROM 13, the storage apparatus 14, the input apparatus 15, and the output apparatus 16 are interconnected through a data bus 17.

The CPU 11 reads a computer program. For example, the CPU 11 may read a computer program stored by at least one of the RAM 12, the ROM 13 and the storage apparatus 14. For example, the CPU 11 may read a computer program stored in a computer-readable recording medium, by using a not-illustrated recording medium reading apparatus. The CPU 11 may obtain (i.e., read) a computer program from a not-illustrated apparatus disposed outside the risk estimation apparatus 1 through a network interface. The CPU 11 controls the RAM 12, the storage apparatus 14, the input apparatus 15, and the output apparatus 16 by executing the read computer program. Especially in the example embodiment, when the CPU 11 executes the read computer program, a logical functional block(s) for estimating a risk related to leave of absence, job separation, productivity or the like (hereinafter, referred to as a “company risk” as appropriate) due to work stress of a subject person (here, an employee) is implemented in the CPU 11. In other words, the CPU 11 is configured to function as a controller for estimating the company risk. A configuration of the functional block implemented in the CPU 11 will be described in detail later with reference to FIG. 2.

The RAM 12 temporarily stores the computer program to be executed by the CPU 11. The RAM 12 temporarily stores the data that is temporarily used by the CPU 11 when the CPU 11 executes the computer program. The RAM 12 may be, for example, a D-RAM (Dynamic RAM).

The ROM 13 stores the computer program to be executed by the CPU 11. The ROM 13 may otherwise store fixed data. The ROM 13 may be, for example, a P-ROM (Programmable ROM).

The storage apparatus 14 stores the data that is stored for a long term by the risk estimation apparatus 1. The storage apparatus 14 may operate as a temporary storage apparatus of the CPU 11. The storage apparatus 14 may include, for example, at least one of a hard disk apparatus, a magneto-optical disk apparatus, an SSD (Solid State Drive), and a disk array apparatus.

The input apparatus 15 is an apparatus that receives an input instruction from a user of the risk estimation apparatus 1. The input apparatus 15 may include, for example, at least one of a keyboard, a mouse, and a touch panel.

The output apparatus 16 is an apparatus that outputs information about the risk estimation apparatus 1 to the outside. For example, the output apparatus 16 may be a display apparatus that is configured to display the information about the risk estimation apparatus 1.

Next, a configuration of the functional block implemented in the CPU 11 will be described with reference to FIG. 2. FIG. 2 is a block diagram illustrating the functional block implemented in the CPU 11.

As illustrated in FIG. 2, a feature quantity input unit 111, a feature quantity normalization unit 112, a model learning unit 113, and a risk degree estimation unit 114 are implemented in the CPU 11 as the logical function block for estimating the company risk. Furthermore, a physiological information storage unit 141, a ground truth data storage unit 142, and a model storage unit 143 are implemented in the storage apparatus 14.

The physiological information storage unit 141 stores a physiological information about a subject person for which the company risk is estimated. Here, the “physiological information” is information indicating an activity status of the subject person (i.e., a human), such as a nervous system and a visceral system. The “physiological information” includes, but is not limited to, an electrodermal activity, a skin temperature, an acceleration (i.e., a body motion), a body temperature, a pulsation, a respiration, a heart rate, a temperature, and an expression, by way of example. The physiological information about the subject person may be obtained by various existing sensors, such as, for example, a camera, a wearable sensor and a smartphone, or devices equivalent to the sensors.

The physiological information storage unit 141 further stores an inner state information indicating an inner state of the subject person estimated on the basis of the physiological information. Here, the “inner state of the subject person” refers to mental and psychological aspects of the subject person. The “inner state information indicating the inner state of the subject person” is information indicating mental or psychological conditions (e.g., joy, anger, grief, pleasure, anxiety, tension, depression, etc.) or information indicating index values (e.g., a stress value, an anxiety degree, a tension degree, etc.) related to an extent of mental or psychological conditions. A “stress level” is an example of the inner state information suitable for the estimation of the company risk (i.e., the risk related to leave of absence, job separation, productivity or the like due to work stress). Since the existing techniques, such as, for example, those disclosed in Non-Patent Literature 1, can be applied to a method of estimating the inner state information on the basis of the physiological information, a detailed description thereof will be omitted.

The feature quantity input unit 111 obtains a physiological information and an inner state information about one subject person from the physiological information and the inner state information stored in the physiological information storage unit 141. Here, the feature quantity input unit 111 obtains a factor information, which is a statistic of the physiological information and the inner state information about factors that supposedly cause a change in work stress of the one subject person during the work of the one subject person, as the physiological information and the inner state information. Note that the “factors” are events that directly or indirectly affect the change in work stress. Such “factors” can be enumerated on the basis of, for example, interviews with the subject person, or with an employee of the same type of industry or occupation as that of the subject person. The “factor information” is numerical data that materialize the status of the factors.

Specifically, when a risk degree of a company is determined by, for example, a factor Ai (i=1, . . . , n), the feature quantity input unit 111 extracts a factor information Bi (i=1, . . . , n), which is a statistic of the factor Ai (i=1, . . . , n) from the physiological information storage unit 141 as the physiological information and the inner state information about the one subject person.

The feature quantity normalization unit 112 firstly obtains a feature quantity in a predetermined time unit (e.g., 1 minute, 1 hour, 1 day, etc.) that is applicable to the estimation of the company risk using a learning model described later, from the physiological information and the inner state information (e.g., the factor information Bi (1, . . . , n)) about the one subject person extracted by the feature quantity input unit 111. The feature quantity is obtained as, for example, a maximum value, a minimum value, an average value, a deviation value, a change amount or the like of each value of the physiological information and the inner state information in the predetermined time unit. The feature quantity normalization unit 112 further performs normalization by an existing method, such as, for example, flooring, with respect to a feature quantity having a relatively large dynamic range out of the obtained feature quantity.

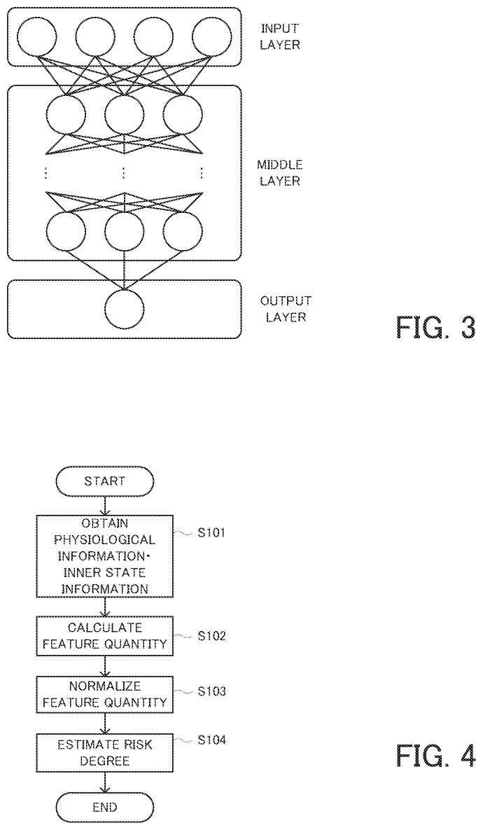

The model learning unit 113 builds a learning model that allows the estimation of the company risk. Specifically, the model learning unit 113 builds a learning model that allows the estimation of the company risk by machine learning (so-called supervised learning) using learning data with a ground truth that are stored in advance in the ground truth data storage unit 142. The built learning model may include a neural network structure with one or more middle layers, for example, as illustrated in FIG. 3. The learning model built by the model learning unit 113 is stored in the model storage unit 143. Since the various existing example aspects can be applied to a method of building the learning model, a detailed description thereof will be omitted.

The risk degree estimating unit 114 inputs the feature quantity obtained by the feature quantity normalization unit 112 into the learning model stored in the model storing unit 143, thereby to estimate the risk degree indicating the company risk for the one subject person.

(Operation)

Next, the operation of the risk estimation apparatus 1 will be described with reference to a flowchart in FIG. 4.

In FIG. 4, firstly, the feature quantity input unit 111 obtains the physiological information and the inner state information about one subject person from the physiological information and the inner state information stored in the physiological information storage unit 141 (step S101).

Then, the feature quantity normalization unit 112 obtains the feature quantity in the predetermined time unit, from the physiological information and the inner state information about the one subject person extracted by the feature quantity input unit 111 (step S102). Subsequently, the feature quantity normalization unit 112 performs normalization with respect to a feature quantity having a relatively large dynamic range out of the obtained feature quantity (step S103).

Then, the risk degree estimating unit 114 inputs the feature quantity obtained by the feature quantity normalization unit 112 to the learning model stored in the model storing unit 143, thereby to estimate the risk degree indicating the company risk for the one subject person (step S104).

(Technical Effects)

In recent years, labor shortages have become more apparent as the population of the productive age decreases due to decreasing birthrate and aging population. Corporate managers are forced to position their employees appropriately in appropriate departments in accordance with the characteristics of each and every employee so as to increase productivity. In the present circumstances, however, an effective method is not established. In particular, securing human resources is an urgent management issue in industries with high turnover rates, and there is an enormous loss caused by employees' leave of absence or job separation. In addition, when there is a person who is on leave of absence or who has left a job, time and financial costs of human support in the surroundings, securing new human resources, and education are very large. For this reason, corporate managers and management departments are required to anticipate or predict various risks for employees (e.g. risks such as leave of absence, job separation, and reduced productivity) at an early stage and to take measures.

It is generally said that a leaver takes more vacations immediately before leaving the job. The leave of absence or the job separation is considered to be predictable by watching a vacation information, but it is usually predictable when the leaver starts to take more vacations, i.e., just before the leaver takes action with the intention of leaving. In addition, when chronically accumulating stress before leaving the job, the leaver often does not take a concrete action, such as taking a vacation.

In the risk estimation apparatus 1, as described above, the risk degree is estimated on the basis of the feature quantity obtained from the physiological information and the inner state information about the subject person. Here, it can be said that the physiological information and the inner state information about the subject person reflect physical and, or psychological burden (i.e., work stress) caused by work of the subject person. The change in work stress of the subject person is considered to appear earlier than the concrete action, such as, for example, suddenly taking more vacations is taken. Therefore, according to the risk estimation apparatus 1, it is possible to estimate the company risk, which is the risk related to leave of absence, job separation, or productivity due to work stress of the subject person, at a relatively early stage.

Especially in the risk estimation apparatus 1, among the obtained physiological information and inner state information about the one subject person, the physiological information and the inner state information about the factors that supposedly cause the change in work stress are extracted by the feature quantity input unit 111. With this configuration, it is possible to exclude the physiological information and the inner state information that are unrelated to the factors that supposedly cause the change in work stress of the subject person (i.e., information that is a noise in estimating the risk degree), and it is possible to improve reliability of the risk degree estimated by the risk estimation apparatus 1.

Incidentally, the “feature quantity input unit 111”, the “feature quantity normalization unit 112” and the “risk degree estimation unit” in the first example embodiment, respectively, correspond to an example of the “extraction unit”, the “arithmetic unit” and the “estimation unit” in the Supplementary Note described later. The “physiological information and the inner state information” in the first example embodiment correspond to an example of the “physical and mental information” in the Supplementary Note described later.

### Second Example Embodiment

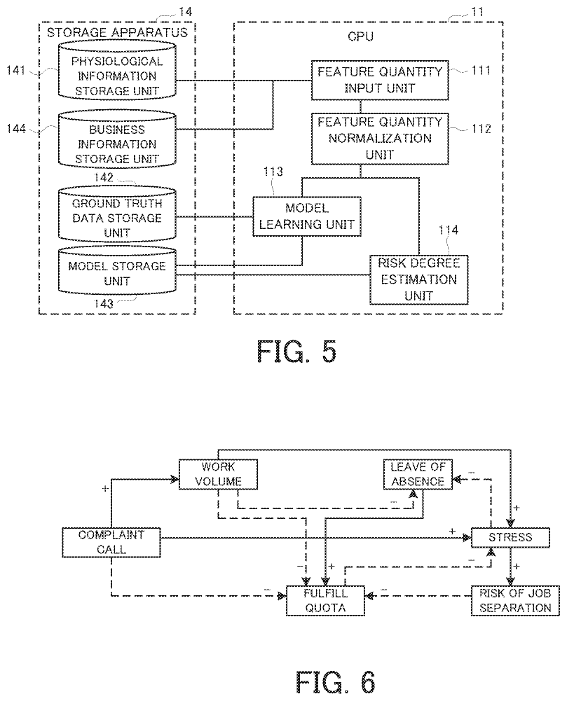

A risk estimation apparatus 1 according to a second example embodiment will be described with reference to FIG. 5 to FIG. 7. The second example embodiment is the same as the first example embodiment described above, except that the operation is partially different. Therefore, in the second example embodiment, the description that overlaps with that of the first example embodiment will be omitted, the same parts on the drawings will be denoted by the same reference numerals, and basically, different points will be described with reference to FIG. 5 to FIG. 7.

(Configuration)

As illustrated in FIG. 5, in addition to the physiological information storage unit 141, the correct data storage unit 142, and the model storage unit 143, a business information storage unit 144 is implemented in the storage apparatus 14. The business information storage unit 144 stores a business information indicating information about a business that causes a change in work stress of the subject person. The business information includes objective information that is closely related to a workload or a business load. Specifically, the business information may include, for example, an objective statistical information such as (i) an attendance information such as working hours, overtime, and vacations, (ii) labor statistics (e.g., the amount of movement (travel distance) and the total amount of baggage in charge for a warehouse worker, the number of calls received and duration of calls for a call center operator, etc.).

It is desirable to use a causal loop diagram, for example as illustrated in FIG. 6, to determine what corresponds to the business information. FIG. 6 illustrates an exemplified causal loop diagram illustrating a causal relationship between a risk of job separation at a call center and the factors lead to it. For example, if there is a complaint call, an operator's workload would increase, and thus, there is a positive relationship between the “complaint call” and the “workload.” On the other hand, when there is a complaint call, a quota is rarely achieved, and thus, there is a negative relationship between the “ complaint call ” and the “achievement of the quota”. In FIG. 6, the positive relationship is represented by a solid line arrow, and the negative relationship is represented by a broken line arrow. By using such a causal loop diagram, it is possible to clarify a background of the risk of job separation, and it is relatively easy to know what kind of information should be watched as the business information. That is to say, when the risk of job separation is obtained as the risk degree, when the factors include a complaint call, a workload, achievement of a quota, vacations, and stress, and an example of each factor information includes the number of complaint calls, working hours, a rate of achievement of the quota, the number of vacations, and a stress level, the feature quantity input unit 111 extracts each statistic from the physiological information storage unit 141 and the business information storage unit 142. Then, the feature quantity normalization unit 112 calculates the statistic in a predetermined unit and performs normalization or the like to obtain the feature quantity to be inputted to the model learning unit 113. Then, the risk degree estimation unit 114 inputs the feature quantity obtained by the feature quantity normalization unit 112 into the learning model stored in the model storage unit 143, thereby to calculate the risk of job separation.

(Operation)

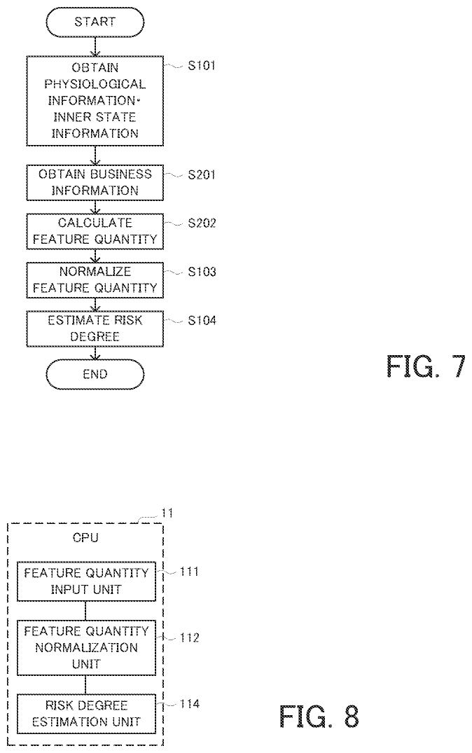

Next, the operation of the risk estimation apparatus 1 will be described with reference to a flowchart in FIG. 7.

After the step 5101 in FIG. 7, the feature quantity input unit 111 obtains the business information about one subject person from the business information storage unit 144 (step S201).

Then, in the step S101, the feature quantity normalization unit 112 obtains the feature quantity in the predetermined time unit from the physiological information and the inner state information about the one subject person extracted by the feature quantity input unit 111, and obtains the feature quantity in the predetermined time unit that is applicable to the estimation of the company risk using the learning model, from the business information obtained in the step S201 (step S202).

(Technical Effects)

According to the risk estimation apparatus 1, as described above, the risk degree is estimated on the basis of the feature quantity obtained from the business information, in addition to the physiological information and the inner state information about the subject person. With this configuration, for example, it is possible to detect an increase in the company risk before a significant change appears in the work stress of the subject person, and it is possible to improve the reliability of the estimated risk degree by complementing and, or reinforcing the feature quantity obtained from the physiological information and the inner state information.

### MODIFIED EXAMPLE

As illustrated in FIG. 8, the feature quantity input unit 111, the feature quantity normalization unit 112, and the risk degree estimation unit 114 are implemented in the CPU 11, but a function block other than the feature quantity input unit 111, the feature quantity normalization unit 112, and the risk degree estimation unit 114 may not be implemented. That is, the learning model may be built by a different apparatus from the risk estimation apparatus 1.

<Supplementary Note>

With respect to the example embodiments described above, the following Supplementary Notes will be further disclosed.

(Supplementary Note 1)

A risk estimation apparatus described in Supplementary Note 1 is a risk estimation apparatus including: an extraction unit that extracts a factor information, which is a statistic of a physical and mental information about a factor that causes a change in work stress of a subject person, from a physical and mental information including at least one of a physiological information of the subject person and an inner state information indicating an inner state of the subject person estimated on the basis of the physiological information; an arithmetic unit that obtains a feature quantity in a predetermined time unit from the factor information; and an estimation unit that estimates a risk degree related to at least one of leave of absence, job separation or productivity of the subject person, by inputting the feature quantity into a predetermined learning model.

(Supplementary Note 2)

A risk estimation apparatus described in Supplementary Note 2 is the risk estimation apparatus described in Supplementary Note 1, wherein the arithmetic unit obtains the feature quantity from a business information indicating information about a business that causes a change in the work stress of the subject person, in addition to the factor information.

(Supplementary Note 3)

A risk estimation method described in Supplementary Note 3 is a risk estimation method including: extracting a factor information, which is a statistic of a physical and mental information about a factor that causes a change in work stress of a subject person, from the physical and mental information including at least one of a physiological information of the subject person and an inner state information indicating an inner state of the subject person estimated on the basis of the physiological information; obtaining a feature quantity in a predetermined time unit from the factor information; and estimating a risk degree related to at least one of leave of absence, job separation or productivity of the subject person, by inputting the feature quantity into a predetermined learning model.

(Supplementary Note 4)

A computer program described in Supplementary Note 4 is a computer program that allows a computer to execute the risk estimation method described in Supplementary Note 3.

(Supplementary Note 5)

A recording medium described in Supplementary Note 5 is a recording medium on which the computer program described in Supplementary Note 4 is recorded.

The present invention is not limited to the examples described above and is allowed to be changed, if desired, without departing from the essence or spirit of the invention which can be read from the claims and the entire specification. A risk estimation apparatus, a risk estimation method, a computer program and a recording medium, which involve such changes, are also intended to be within the technical scope of the present invention.

This application is based upon and claims the benefit of priority from Japanese Patent Application No. 2019-111762, filed Jun. 17, 2019, and incorporates all of its disclosure herein.

### DESCRIPTION OF REFERENCE CODES

1 . . . Risk estimation apparatus, 111 . . . Feature quantity obtaining unit, 112 . . . Feature quantity normalization unit, 113 . . . Model learning unit, 114 . . . Risk degree estimation unit, 141 . . . Physiological information storage unit, 142 . . . Correct data storage unit, 143 . . . Model storage unit, 144 . . . Business information storage unit

What is claimed is:

1. A risk estimation apparatus comprising a controller,
the controller being programmed to:
extract a factor information, which is a statistic of a physical and mental information about a factor that causes a change in work stress of a subject person, from a physical and mental information including at least one of a physiological information of the subject person and an inner state information indicating an inner state of the subject person estimated on the basis of the physiological information;
obtaining a feature quantity in a predetermined time unit from the factor information; and
estimating a risk degree related to at least one of leave of absence, job separation or productivity of the subject person, by inputting the feature quantity into a predetermined learning model.

2. The risk estimation apparatus according to claim 1, wherein
the controller is programmed to obtain the feature quantity from a business information indicating information about a business that causes a change in the work stress of the subject person, in addition to the factor information.

3. A risk estimation method comprising:
extracting a factor information, which is a statistic of a physical and mental information about a factor that causes a change in work stress of a subject person, from a physical and mental information including at least one of a physiological information of the subject person and an inner state information indicating an inner state of the subject person estimated on the basis of the physiological information;
obtaining a feature quantity in a predetermined time unit from the factor information; and
estimating a risk degree related to at least one of leave of absence, job separation or productivity of the subject person, by inputting the feature quantity into a predetermined learning model.

4. (canceled)

5. A recording medium on which a computer program is recorded,
the computer program allowing a computer to execute a risk estimation method,
the risk estimation method comprising:
extracting a factor information, which is a statistic of a physical and mental information about a factor that causes a change in work stress of a subject person, from a physical and mental information including at least one of a physiological information of the subject person and an inner state information indicating an inner state of the subject person estimated on the basis of the physiological information;
obtaining a feature quantity in a predetermined time unit from the factor information; and
estimating a risk degree related to at least one of leave of absence, job separation or productivity of the subject person, by inputting the feature quantity into a predetermined learning model.
